SEQUENCE LISTING (27 sequences)

SEQ ID NO: 1 - DNA, 10006 bp, Homo Sapiens
cccaaattgtctatctgtgatagtggctgtgccccttcgggccctgagcaccctgtgtcctgtgcagcagtcagatatctggagggagactgaggcactggctgcagagcttgtgatcatgagagagactcactaggactacagatgggtaaactgaggccttcgagggggcagctccagaaaggcaggggccataatgtctcaccttcatatttcccgtgccaagctgtggccttctgcattcatggcagatgagtggacaaaggctgatggactgatggagaaacaaagggatagatggagcagctgggcagctcagcaaatgatgctgcaatgatctgcttccaactcacctcaaatccatccttctctctccaggcagagtgggcttttaagatacacatctggccaggtctctcactgttcaaacccttcatctgctcctttttgccttcaggataacatcccaccctcctatcaaggactatggagccctgtgggatctggttcccacttgattctccaacttcctcttcccctatgccctgccttctcatctgttccagtgctattatgaagccacacgttcttcctttattatcaagcataccacagtttatctcacctcagaggctttgcacagtatattttcctagggaggggtccccaggtggtagaaaaacggttacagccaactcctccatgtgtcactcaagacccttcaacagcaggctgcagatttcctctccagcactgtgctccaactgtgtaatggatttctgtgtctgcctccttgactcaacccaaatgaacaagagccatctatctctgtatctctgcaatcacaggcacaaaataggtgctctctacattttttccaacctgagaggccattctagaagggtctcaggccacggttctgtccagtattccatgcagatgctgacaggactgcaattaaaaaaatacttgagatgcccaaatgcccaaatagcttctcattttgctttgactaccaataattgcacagtgcaatagaataatgctcaaatacattaacatcttacttgatcctagggggtcctctctacttttaaagcctcaaacttcctccctctcacaggtgaaaaggggagtacaaatacattccctcccttgctctgcggatccattcctacaggtagtcaagactctgagcttcccctctgactttctggcagtgcttcacctctccccacagatgagtgcaggaacaattctaacagacttcagactcttccaacagagccaatccctccccatcacgttggagtggacttgctacccacaccatcaacaggcccctgagaaacttcacagggcaggggcttctttggtaaaaccaaccctttccttccaccagctcagagaagttgttccagatagatgccaggattcttggaggagcatggtgattctggggtggagcctctgaccctggccaaccaaaggcctgagccactcttcccccacaaagtgattggcgcaggagtgggcatgtaagctggacccagccaatcagcataaccaaatcccttgcccaccctggggagggtcctatagttgttgccaagagaggctcacccctgttctcaagaagcttacaactggtaggggcaacaggttttaatctatcattacacaaatatttaatttcaatactgttgcaataagagctatgaaggaaaagaacactccccctacttttacttggtgttaagatttgaagaagaaaaaaaaaaaacactgcctgaaggattgttatggccttctatataatagtggctgcagacatttgcccattatgttcagcatgaacccatgtgacaaattcatcaaagcgttttgcactagggagaaaaatttgtattagaggaagcacagcagtttggactgaaagacaaagaaaattcagccaattctgctgatcttttttgatggggcacctggaagctgaaagctaaagtggtactcaggaacagggactgctacttctgttcctggtgaatcctgccccaaacctcctctctctctgattcctgattccactgtgccagtgggaatatatgctccccaagatgtcaaaactaaagggaaattgcaaaaaatatatacatatatttttagagagaaaataagattataaaaaatgtgttttgtaccccccaagtttcactaagaacttcctgacttccaggccctggttgtgccccacgcaccagcctgcccagctttcctggaccaaacttcctagcacctaagcaggggatgagggcagataaactaaatcagaaaagggatctgttcctcctagactcaaccaacatgaccaccgtggggaaagaagaaacaaaaacaagagcaaactctcttaaagagcagcctggcagctatcaccattagcattcagcccctctgtccacaggactcaggaccaaacccctcaccttcactatcccatccgtttcccaagaagcagaaatacttattctcacatttcacagatggggaagctgaggctaggagaggttatgttatttgccaaagccacacaactagtaaaagccactgacaagattctggctcaggccatcaggtgccagaggcagcattttttggcaccacaggccctgcctgggaacaagagcatgcagaaaatctcacaagagatgggaacaaaatttggaaaattgctagcgtgcagggaggggggaaggtgtgatttcctgctacagacgccagagtaaaagccaccccaggagtgcctgtgcagccctccatagtaaggtccagcggctgcatttatgcccaaagatgcccctggtgcttggagtggaaggaagattccagagacaagattagaaacttctcagcttagcagctctagggctggacccgccaacaagccattttacacataaagcagtcaatgggagggggtagacgtagggggctaaactccccacagcacagggtccaagttggtagactgcactttctccaggcgcaggtccgctagtgccggcatcggggactcgttatcttaacttgcgaccctgggtgcacagagccctgcacacaccactggagaggggttcctgctgtcgagggttgagaggagggtatggagtccctggaacagcacgacagggtgcagaggccacctggcagggcctgaacaccgaggcctctgtgagcttgggtcgggccggcttcccgcttcggaggttggggagggggtcgtgggtctctgcgttcccaggccaagcggccctggaggcacggactagcaggacgccgaggtggcgcgggtcgcggcctctcccgcagcagctgtcggcgagaaccaggcagggaggcgccgctgctgactgcatctcatgaaagattcaggcccggctgccgcgctgccatctcccggcaccttgcgccggaaacggtcgctctggagcccgtggccgtcggcgggcaggcttagccgctccagtccctgagaaaggcaggccacagcccgacctgccctgtggtcccatcccataatcccaacagcaagcaggctcaggctgggcacttcggggtaccaggagtaggttcggccaactggtttccaccatgaggcttcgcgcacagggttatctggccacgaggcaacgctggggagccctgtggcctgagggtgggcaaaggacaggctcccagttcccttgcgtccagcccgtctcccagcggcagccagccaggaacggcctgcggggccacaggggtggaggcgtcaccgttcgcaggcccgcagcaggatggtcgctgggggatgtgcaggcataggggttggacaaggggccccagaagtgtctgtcctgaggggttggtgtgccctttcctcaccagcccagcccctgaggagagggaagaaggcaattcccccaaccagggcaggtcgggcggttgccccaccctaacaccccctacccccaaacacagaaaacctggggtctgtcctcaaacctccctggctgccacgctctgggcagcggactctccctgccaacacgcaagaccagctccctcccgcaggctgagcagaaagaagaaaggtccaatctcaaaaccccaaactcgaccaccagcccccgtctaaacggaagtagggccagcccctcacgagtcaggtgggaaactgggcccagggagagaaagtgccccagggccaggctgggacccgctctggcttcgtccgcatgcggcagggcccctccacggaggtcccagggcgcgctccccggcctcgaggcccggccgccagccgcgcggaccccagcctacgccccgagggaggccaggacccctagccggcgggactgcgcgccgcccctctccccgcaggtcccggcgaacacctagcttcccctccccccacccttcccgcctcccggccagtgtccccgccttccccgcgggcgacgggcggcggcggcgggaggagcgggccgagccgaggaagccccggcctcgcgcgctgggatgtagcgaaccagcaggggccgaagaaccgtgcagtgccagagccagagctggatccggggccccagccggagccgaaacctgagccagagtccgcggcgggcgagcccggagcccacgagccgcagacgcagcgctgcccaggtggggtaagagacgctgggctaggggcgcagggtctccgcggtggaggggcgcagggaggtggcggccgagtcctgcgcagtttgctcctggcgtgtgtgggtccacccggcggcgcgggacagcgcaaggcgcggaaggtcaggagccttcgaggcagcgcgaggagctcgttcctgcgcccagggcacagtcatagccgccgtcaccgggtgctacctcacccaaccggcgggatcaaccctctgctttggctccgggcacctcaagagggtagcagcctcgggggcacgggccacggccccgcgaagggcacaacctgagaagcccgtggcagcccctcgcagcgtcgggtgacacagggctcccccacccccaggagaagtgggcaggagagagggccgcccgctgctccccgctgcgtccagggatggagggccccaccacccatggaattgctggcccctctgcgtggcccgggacttcagccgtggcttcgcgtcaagagggtatttttcctaaacgaaaccgcttcgttcgttcgttcgttcgttcgttcgggcagcaatgccgcagaaaagcagcagacgtcggtccgcgccctggctctcttcgccccggaccccgacgtcccgccgcagcgctcggaggtgcccccagcccaaggcagcctgctctcgccggcacaggtcgggctttttcttcccaggagagaaaccccaattcccttcgtaacgtccaataaagacattcccgcggcttctcccaggtttggttgttgacgcagggtcccggagcacgcagtcgcttctcaagaaccgggtctcggatttctgaaattgaccagcttcgtaaattggagcctattctcccgcggcaaaggcagggccccaaagccgggatcgcagtaatgggaaccccaggctggaatccgggtcccaagcttttccgatttaggaattccccgaatctacaaatatttagtccacttttctgaaaaactaaattctgaaaaacacaaattctcttgacatccctgtgacctctgaaagccaccagggccagagggaggaaatcccaggttgctgtccactgggggaggattcaggtctagggttcaggtctacggtagtcagggcaaaagctacaggcagcaggggcagcacaggagacttgctgtccccgtgccctttcccggggctgctttcggcctcccgcatctcttccagggaaaggaaaagaggtgggctggggcttggagaccaggctgtctggactctaggatgcagaggcctccagacaggctcagggtgctcttctcccatgaaagcagccgctgggaggaggaggctatggtgcatccataagttgcccctctgctccccagttgtgcgaccagctgctacctccttcctagtcttcttccccacagctcagccattctcaggaaccagacagcgtccatggacttaggtgagagatgggccgggtagagccatgggtcctaccagccgctgactgagcggcccacggcacagagtcctgagttccatactcccatctgtgcctcactggcggcagtcctgctcaaatacatcctggctctccccgggacaggctggggatccccatttggcaggaagcctcagactggggtcccaggaagcctaaaggagccagtgaggtctttccagcccctacctgagcaccctcctccccacttacccagtaattgctgtattcaaagaaacgggagcttttattggggagggggtgttagatcaggcagaaagaggtaggtggtccaaacctgcactcccaaaacagggttttcaagtttgaacttctccacggactaagaggcttagggctggaatgtcccagagagtcatggatagccctggtggcaggccatggcacattccttcctttttcctaaaataccttgattctgggagcaaggattagggcacggtgcccccgtgggtgggtagaaggatgcccccccactgagagccttccaaccacccttcccaaattacattactaaaccattcttgggcacagggtgtttttagtgagccaggcttcaggaagggtcctcatggtgactacttcaaccccacaacagcccaagctcttctgctcagcccagccaagaccctaaactccaaaattcttgaaaatcagagaatcattgctggctttgtgtggtcacggaggggtggggaacagggcacatggttccagctccactaagcccccttccctcctctcttcgtgtcccatcagcaagtgagctggatgctgaagcagcaggcagagtccggtgttggacatgggaactgaggcacagtgcagatcaagccttaaccttgagggaaacacaggtcacatagcacagctgggggaacacaaagcctctgcttactcctgaaagagtgctgttttctgtcctgtatgtgtgacgtgtctgtgagcgtgcaagaagcccctatcttgactgggacaatggccagtgagtgtagctggggaagaattgagagcatgtccaggtcccttccccagccaacgcccaagatcaggccacagcctcctcacaatcaattgcctcctcactccttgatcactcagtgctgcccaggcccagcagaacagactctgccagcaggccccactagccccagctcctctttgggtctcaggtcccctgaggatatggggcttcacctgaaatggtctgagggcttttccttctacacagcaggcatcaagatcaccaaataaagggactattgtgcctgcctggagccctgccagaggtttgggcccagaggggcacacagcaggtgctcaataactgcattaaatgcactaacagtgaggaaacacgcccctcagactaagcagtgagtgctgctcacagaatagtccccattgggggatggcccaaagagtcactttggtccctctgggaagtgagaaggcaagtgagaaggctgtgagtcttaacctcctctagaggcccacagacagaccattcatttctaagtctctacccagagacgcactgtgcttcccaccttggcctgacatgtggcagggttagaacacacctcctatcccctgccagcccgcgttcatgccaagtagcacatatatgcctaaactcagcacttccatagtgcagtgaatacatgtgtgtgtacagcatctccgcatggatgtacaggatgtgtgtgtgtgtgcgtgcccccatgctgtctgcattcaggctgttctttttggtaagacagctaaaaaaagaatggtctgtgaagggacactccctagcacgctgcaacacctgaatatctccttgaaaggagggatcttctactgcaggagactcgtggtaaaggtggccaagaaacatggcaacggtggggctgagggcaaatgctgggcaactgtgcttccccatgttcccctccccgtagccaagactcatttcatggagggagatctcagcttggaagaaggcaggagtcactgagcctccccaatccaaacccctgagaagtgtcctccctctggcctcagaccctgcatcctgtggtcacagacccacagtgagaaaggaccaggccctaaggagctgtgctgtctctccacggcccagagcgggggatggggatggggatggggatggggatggggatggggatgggggtaggggtgggggtgctttggactaacgtggagggaatggaaggcaggcctggttccaccctgcatgcccgaccctggccccagcagcccccacaaggagctcagctgaccctgggtgtctccctgtgatgggaaggggtaagacgaggactcaaaggcagaacctgcagagtgccccagacgctgatacctgcacagtcagtgccacccacccaggagttgaggaggcactgggttttggggtgaggacactggacacctccctgcttctttcccaggcagacaatcctggcgcagctcccttgggttgctgtgtctggtggagctgatcacaggtgaggggcagagggcagtctggggtccgcctatggccagaggagcaggtcagggcggcgccttgccgccccagctgtggcctgtttgctcaagacgctgaggtctcggggccagctaacaattgttgagcaaaatccttcgacaaacttcacctacgtgcaaggactcgaggagggaatcactcttaggagtgggagagtaatgtctttgcctgtgcccagtgaaggcccattggagctgcagctcagctaccactgtgtgggagagaagctggaagactgagggcttcctgggctgctggcccagggttgggagacagcagtcacctggcttaccaggcctatgcctgaagccctgggaagccaggacgcaggccccaggctgggacaaagctaccctgaaggagggcaaaggctgccaaggccaaccccatgcctgccaaggccaggcctggcccatttggccaaggcctaaggtgtaaaacaaggggagaggtacaagaggctgtggggtctggctgggatccttggggtcttccttctgcattctccaaacgcctagagccagcagaaacgtttcgtctgattagaagccatcatttctatcccaatcccggaaaattgactgcggtgcagagagggaggcctgagaagcagccgtaggggagaaggtccaagctaattaggaggcagcatccgggggcccattagagcgcaggctgctgtcactcagccgggctgagttcccgggagaagaggctggagaaggaggggcaggcggcccctcgacgaggacaccgctgggagctgccggaacgggccccgggctctgcccccgccccggcgctggctcgaaggcgcccgctcggtgcgatcctgttcggcaaacattcactcatcctgggctgttctcgccagggctggggacttcgaggcggccgagacgggagttgattctaggcgaaacaagtcatttgaggcctgaggtgtgcacgagccgcccgggactcgcaggccagatgcgtttcttttgtgaggccgagggagaactcggtgtgtcaccggggaaggagggagaggcgcggcgaggccgcggggggcggggaggcggcgggaaggtggctgcggagggggagggcgcgggcgaggcagggagggagggagggcggcagtgagggcgcggcggcgcgggcggcttggggctggattccgcccgcgctccctcgctcgctcgctccctccccagccccctccca

SEQ ID NO: 2 - DNA, 2373 bp, Homo Sapiens
cccaggcccaccccacccagcacccctggcgcagggactgctggaacctggctgtgcgcgctgtcgctttaagacagactctgccggcgccgtccggagccttagaaaccggccccggatcgcgagccggagccggagccggagccggggccggccgggctgctgaggcccgagcggcaggagcgcagcgcggagcgctgagccaggcgcccagtcgcgagaagctgccgccgcctctgcccgcccggcgccgcagccccgggcggtccatggggcgggcacggcgtcgctgcaggcgccggcagccctggagggcagccgcttaggcgctgcgctcttgtccccgcaggtcgcagccagggcggcggggcgcgcccagccccggcccctggagcgcccgccgcggtccccacctccatggacgccttcaaggggggcatgagcctggagcggctgccggaggggctccggccgccgccgccgccaccccatgacatggggcccgccttccacctggcccggcccgccgacccccgcgagccgctcgagaactccgccagcgagtcgtctgacacggagctgccagagaaggagcgcggcggggaacccaaggggcccgaggacagtggtgcgggaggcacgggctgcggcggcgcagacgacccagccaagaagaagaagcagcggcggcaacgtacgcacttcacaagccagcagttgcaagagctagaggccacgttccagaggaaccgctaccccgacatgagcatgagggaggagatcgccgtgtggaccaacctcaccgagccgcgcgtgcgggtctggttcaagaaccggcgagccaagtggcgtaagcgcgagcgtaaccagcagctggacctgtgcaagggtggctacgtgccgcagttcagcggcctagtgcagccctacgaggacgtgtacgccgccggctactcctacaacaactgggccgccaagagcctggcgccagcgccgctctccaccaagagcttcaccttcttcaactccatgagcccgctgtcgtcgcagtccatgttctcagcacccagctccatctcctccatgaccatgccgtccagcatgggcccaggcgccgtgcctggcatgcccaactcgggcctcaacaacatcaacaacctcaccggctcctcgctcaactcggccatgtcgccgggcgcttgcccgtacggcactcccgcctcgccctacagcgtctaccgggacacgtgcaactcgagcctagccagcctgcggctcaagtccaaacagcactcgtcgtttggctacggcgccctgcagggcccggcctcgggcctcaacgcgtgccagtacaacagctgaccgccccgccgcaccacgcgggccggcggccggagcggggaagggcgcgggcgcggaggacgcacgcggggccccggctcgcaagccccagctcaccgcgccgcggacctcacacctgcgcagccccctcctcccacttcccactccgggttggttttgtgtttgcttttccggaccccactctgccctccaaaaagacaaaaaaaaaaaaaaaaaaaaaagcaaaaagacgtcggagaaaagtgccgcgaaaaaatggatgagttgcaatttctctcgggatggcgcgggtggtgtgtgtgtgttcccacgggccccggaggcccactccgcggagggcacgcggcgcggtaggcgagcgccgaggcccagcggccgggggaggacgacctcgtatcccgcgtccccgccgcgctggatccggactgagtggccgggcctgcggactggatgtgcggggcctggacttgcctaggatttcccgaccccgtacaaaccaagttgccctctccgagctaggcccggccgagagcgccttagctcgagtcggatccgtgttggggcgggcgttgggtttggggggacggtgcccccagcccaggatcgggcactcagtggagccgcacacggccccggcgcgcctggtagagcctcgctggccccgcgccccggagccctatattaaggccacggagcgacagcgggcagtgcgggcctggcgggaggtgggggaggtccatctcagaacaccccagccttgagcttagctgcaggcccaggccctctgctctgctcccgggctaggaggtggccctctgtctgggcgaacagccccctcctcaccgcccgccgtgcaagagtcgagccggcagagcaaggggcgcggccccagggccctgcgcccactttgcacacccgctctccggcccgcgcccctgtttacagcgtccctgtgtatgttggactgactgtaataaatctgtctatatcgactaaaaaaaaaaaaaaaaa

SEQ ID NO: 3 - protein, 314 aa, Homo Sapiens
MDAFKGGMSLERLPEGLRPPPPPPHDMGPAFHLARPADPREPLENSASESSDTELPEKERGGEPKGPEDSGAGGTGCGGADDPAKKKKQRRQRTHFTSQQLQELEATFQRNRYPDMSMREEIAVWTNLTEPRVRVWFKNRRAKWRKRERNQQLDLCKGGYVPQFSGLVQPYEDVYAAGYSYNNWAAKSLAPAPLSTKSFTFFNSMSPLSSQSMFSAPSSISSMTMPSSMGPGAVPGMPNSGLNNINNLTGSSLNSAMSPGACPYGTPASPYSVYRDTCNSSLASLRLKSKQHSSFGYGALQGPASGLNACQYNS

SEQ ID NO: 4 - DNA, 18 bp, Artificial Sequence
  primer
ggaaatcgtgcgtgacat

SEQ ID NO: 5 - DNA, 23 bp, Artificial Sequence
  primer
tcatgatggagttgaatgtagtt

SEQ ID NO: 6 - DNA, 20 bp, Artificial Sequence
  primer
gacccagccaagaagaagaa

SEQ ID NO: 7 - DNA, 22 bp, Artificial Sequence
  primer
gaggttgttgatgttgttgagg

SEQ ID NO: 8 - DNA, 20 bp, Artificial Sequence
  primer
ctgtttgctcaagacgctga

SEQ ID NO: 9 - DNA, 20 bp, Artificial Sequence
  primer
ctcggcctcacaaaagaaac

SEQ ID NO: 10 - DNA, 20 bp, Artificial Sequence
  primer
tgtctgcattcaggctgttc

SEQ ID NO: 11 - DNA, 20 bp, Artificial Sequence
  primer
gattccctcctcgagtcctt

SEQ ID NO: 12 - DNA, 20 bp, Artificial Sequence
  primer
caagtgagctggatgctgaa

SEQ ID NO: 13 - DNA, 20 bp, Artificial Sequence
  primer
agggagtgtcccttcacaga

SEQ ID NO: 14 - DNA, 20 bp, Artificial Sequence
  primer
gctcagccattctcaggaac

SEQ ID NO: 15 - DNA, 20 bp, Artificial Sequence
  primer
gccattgtcccagtcaagat

SEQ ID NO: 16 - DNA, 20 bp, Artificial Sequence
  primer
tcgcgtcaagagggtatttt

SEQ ID NO: 17 - DNA, 20 bp, Artificial Sequence
  primer
taggacccatggctctaccc

SEQ ID NO: 18 - DNA, 20 bp, Artificial Sequence
  primer
cacgagtcaggtgggaaact

SEQ ID NO: 19 - DNA, 20 bp, Artificial Sequence
  primer
gacgtctgctgcttttctgc

SEQ ID NO: 20 - DNA, 19 bp, Artificial Sequence
  primer
aggcacggactagcaggac

SEQ ID NO: 21 - DNA, 18 bp, Artificial Sequence
  primer
atgcggacgaagccagag

SEQ ID NO: 22 - DNA, 20 bp, Artificial Sequence
  primer
ttagcattcagcccctctgt

SEQ ID NO: 23 - DNA, 21 bp, Artificial Sequence
  primer
ttcatgagatgcagtcagcag

SEQ ID NO: 24 - DNA, 20 bp, Artificial Sequence
  primer
acaactggtaggggcaacag

SEQ ID NO: 25 - DNA, 20 bp, Artificial Sequence
  primer
tgtgtggctttggcaaataa

SEQ ID NO: 26 - DNA, 20 bp, Artificial Sequence
  primer
gcactgtgctccaactgtgt

SEQ ID NO: 27 - DNA, 20 bp, Artificial Sequence
  primer
gggggagtgttcttttcctt

(19) United States
(12) Patent Application Publication
(10) Pub. No.: US 2013/0029871 A1
(43) Pub. Date: Jan. 31, 2013

(54) METHODS OF STRATIFYING ADOLESCENT IDIOPATHIC SCOLIOSIS, ISOLATED NUCLEIC ACID MOLECULES FOR USE IN SAME AND KITS USING SAME

(71) Applicant: Chu Sainte-Justine, Montreal (CA)

(72) Inventor: ALAIN MOREAU, MONTREAL (CA)

(73) Assignee: CHU SAINTE-JUSTINE, MONTREAL (CA)

(21) Appl. No.: 13/633,338

(22) Filed: Oct. 2, 2012

Related U.S. Application Data
(63) Continuation of application No. 12/531,740, filed on Sep. 17, 2009.
(60) Provisional application No. 60/895,490, filed on Mar. 19, 2007.
(60) Provisional application No. 60/908,417, filed on Mar. 28, 2007.

(51) Int. Cl. C12Q 1/68; G01N 33/566; C40B 30/04

(52) U.S. Cl. 506/9; 435/6.11; 435/6.13; 435/6.12; 435/7.1

(57) ABSTRACT

A method of stratifying a subject having adolescent idiopathic scoliosis (AIS) comprising: providing a cell sample isolated from the subject; detecting Paired-like homeodomain transcription factor 1 (Pitx1) expression in the cell sample; whereby the results of the detecting step enables the stratification of the subject having AIS as belonging to an AIS subclass.

### CROSS REFERENCE TO RELATED APPLICATIONS

This application is a continuation application of U.S. patent application Ser. No. 12/531,740, now pending, filed on Sep. 17, 2009, which is a National Entry Application of PCT application No. PCT/CA2008/000524 filed on Mar. 19, 2008 and published in English under PCT Article 21(2), which itself claims priority on U.S. provisional application Ser. No. 60/895,490, filed on Mar. 19, 2007, and on U.S. provisional application Ser. No. 60/908,417, filed on Mar. 28, 2007. The contents of the above documents are incorporated herein by reference in their entirety.

### FIELD OF THE INVENTION

The present invention relates methods of stratifying adolescent idiopathic scoliosis, isolated nucleic acid molecules for use in same and kits using same.

### REFERENCE TO SEQUENCE LISTING

Pursuant to 37 C.F.R. 1.821(c), a sequence listing is submitted herewith as an ASCII compliant text file named 14033_{—}103_ST25.txt, created on Oct. 1, 2012 and having a size of 23 kilobytes. The content of the aforementioned file is hereby incorporated by reference in its entirety.

### BACKGROUND OF THE INVENTION

Spinal deformities and scoliosis in particular, represent the most prevalent type of orthopaedic deformities in children and adolescents (0.2-6% of the population). Published studies suggest that one percent to six percent of the population will develop scoliosis. This condition leads to the formation of severe deformities of the spine affecting mainly adolescent girls in number and severity.

At present, the cause of adolescent idiopathic scoliosis (AIS), remains unclear (Connor J M, Conner A N, Connor R A, Tolmie J L, Yeung B, Goudie D. Genetic aspects of early childhood scoliosis. Am J Med Genet. 1987; 27:419-424; and Machida M. Cause of idiopathic scoliosis. Spine. 1999; 24:2576-2583) and there remains a need to stratify children or adolescents having AIS, identify children or adolescents at risk of developing AIS and identify which of the affected individuals are at risk of progression.

It has been showed that Pitx1 +/− mice developed severe spinal deformities after weaning. Paired-like homeodomain transcription factor 1 (Pitx1, previously called Ptx1) is a homeodomain transcription factor detected initially throughout pituitary development. The Pitx-family contains three related members, Pitx1, Pitx2 and Pitx3, which are members of the paired class of homeodomain proteins. The three Pitx factors have similar transcription properties (Drouin, J., Lanctôt, C., & Tremblay, J. J. La famille Ptx des facteurs de transcription à homéodomaine. Médecine/Sciences 14, 335-339 (1998); Drouin, J., Lamolet, B., Lamonerie, T., Lanctôt, C., & Tremblay, J. J. The PTX family of homeodomain transcription factors during pituitary developments. Mol. Cell Endocrinol. 140, 31-(1998); and Lanctôt, C., Lamolet, B., & Drouin, J. The bicoid-related homeoprotein Ptx1 defines the most anterior domain of the embryo and differentiates posterior from anterior lateral mesoderm. Development 124, 2807-2817 (1997)). Among others, this transcription factor controls the development of craniofacial and hind limb specific structures in mammals. The pitx1 gene is highly expressed in mouse hind limb long bones during development and accumulation of high levels of Pitx1 proteins were detected by immunohistochemistry on hind limb long bone sections mainly in the periarticular region, along the perichondrium (including at the hip and knee joints) and also in the nuclei of proliferative chondrocytes (Lanctôt, C., Lamolet, B., & Drouin, J. The bicoid-related homeoprotein Ptx1 defines the most anterior domain of the embryo and differentiates posterior from anterior lateral mesoderm. Development 124, 2807-2817 (1997)). Pitx1 expression was also detected in craniofacial structures such as the mandible and at the temporo-mandibular joints. It has been shown that targeted inactivation of the mouse pitxl gene severely impairs craniofacial and hind limb development. While null mice died at birth, all PITX1 +/− mice which are normal at birth, developed severe spinal deformities (100% starting at 2 months).

The present description refers to a number of documents, the content of which is herein incorporated by reference in their entirety.

### SUMMARY OF THE INVENTION

In accordance with the present invention, there is provided a method of stratifying a subject having adolescent idiopathic scoliosis (AIS) comprising: providing a cell sample isolated from the subject; detecting Paired-like homeodomain transcription factor 1 (Pitx1) expression in the cell sample; whereby the results of the detecting step enables the stratification of the subject having AIS as belonging to an AIS subclass.

In accordance with another aspect of the present invention, there is provided a method of stratifying a subject having adolescent idiopathic scoliosis (AIS) for a clinical trial comprising: providing a cell sample isolated from the subject; detecting Paired-like homeodomain transcription factor 1 (Pitx1) expression in the cell sample; and stratifying the subject for a clinical trial based on the results of the detecting step.

In accordance with another aspect of the present invention, there is provided a method for predicting a risk for developing adolescent idiopathic scoliosis (AIS) in a subject comprising providing a cell sample isolated from the subject; and detecting Paired-like homeodomain transcription factor 1 (Pitx1) expression in the cell sample; wherein an absence of Pitx1 expression is indicative that the subject is at risk for developing AIS.

In a specific embodiment of these methods, an absence of Pitx1 expression is indicative that the subject is at risk for developing a Cobb's angle of 45° and above. In another specific embodiment, the detecting step is performed with an isolated nucleic acid molecules specific to a Pitx1 transcription product. In another specific embodiment, the isolated nucleic acid molecule is detectably labeled. In another specific embodiment, the detecting step is performed with an antibody that binds specifically to Pitx1. In another specific embodiment, the cell sample is selected from the group consisting of an osteoblasts sample, a chondrocytes sample, a skeletal myoblasts sample and a blood sample. In another specific embodiment, the cell sample is an osteoblasts sample. In another specific embodiment, the method further comprises a step of selecting a preventive action or a treatment in light of the results of the detecting step. In another specific embodiment, said subject is pre-diagnosed as being a likely candidate for developing adolescent idiopathic scoliosis.

In accordance with another aspect of the present invention, there is provided a method of selecting a compound potentially useful in the treatment of adolescent idiopathic scoliosis, said method comprising the steps of (a) contacting a test compound with at least one cell known to express Paired-like homeodomain transcription factor 1 (Pitx1); and (b) determining Pitx1 expression level; wherein the test compound is selected if Pitx1 expression level is increased in the presence of the test compound as compared to that in the absence thereof.

In a specific embodiment, said cell is an osteoblast. In another specific embodiment, said cell is from a subject having adolescent idiopathic scoliosis (AIS). In another specific embodiment, the subject is a human.

In accordance with another aspect of the present invention, there is provided kit comprising an isolated nucleic acid molecule specific to a transcription product of a Paired-like homeodomain transcription factor 1 (Pitx1) and instructions to use the probe to predict whether a subject is at risk for developing adolescent idiopathic scoliosis.

In accordance with another aspect of the present invention, there is provided kit comprising an isolated nucleic acid molecule specific to a transcription product of a Paired-like homeodomain transcription factor 1 (Pitx1) and instructions to use the probe to stratify a subject having adolescent idiopathic scoliosis.

In another specific embodiment, the kit further comprises a container for a nucleotide sample from the subject.

In accordance with another aspect of the present invention, there is provided kit comprising an antibody specific to a Paired-like homeodomain transcription factor 1 (Pitx1) and instructions to use the antibody to predict whether a subject is at risk for developing adolescent idiopathic scoliosis.

In accordance with another aspect of the present invention, there is provided kit comprising an antibody specific to a Paired-like homeodomain transcription factor 1 (Pitx1) and instructions to use the antibody to stratify a subject having adolescent idiopathic scoliosis.

The articles “a,” “an” and “the” are used herein to refer to one or to more than one (i.e., to at least one) of the grammatical object of the article.

The term “including” and “comprising” are used herein to mean, and re used interchangeably with, the phrases “including but not limited to” and “comprising but not limited to”.

The terms “such as” are used herein to mean, and is used interchangeably with, the phrase “such as but not limited to”.

As used herein the terms “likely candidate for developing adolescent idiopathic scoliosis” include children of which a least one parent has adolescent idiopathic scoliosis. Among other factors, age (adolescence), gender and other family antecedent are factors that are known to contribute to the risk of developing a scoliosis and are used to a certain degree to assess the risk of developing AIS. In certain subjects, scoliosis develops rapidly over a short period of time to the point of requiring a corrective surgery (often when the deformity reaches a Cobb's angle ≧50°). Current courses of action available from the moment AIS is diagnosed (when scoliosis is apparent) include observation (when Cobb's angle is around 10-25°), orthopaedic devices (when Cobb's angle is around 25-30°), and surgery (over 45°). A more reliable determination of the risk of progression could enable to 1) select an appropriate diet to remove certain food products identified as contributors to scoliosis; 2) select the best therapeutic agent; and/or 3) select the least invasive available treatment such as postural exercises, orthopaedic device, or less invasive surgeries or surgeries without fusions (a surgery that does not fuse vertebra and preserves column mobility). The present invention encompasses selecting the most efficient and least invasive known preventive actions or treatments in view of the determined risk of developing AIS. The present invention also encompasses stratifying AIS patients with methods of the present invention.

As used herein, the terms “severe AIS” refers to a scoliosis characterized by Cobb's angle of 45° or more.

As used herein, the term “Pitx1 expression” is used to refer Pitx1 transcription and/or Pitx1 translation. In a more specific embodiment, Pitx1 expression refers to Pitx1 transcription.

As used herein the terms “risk of developing AIS” and “risk of progression of AIS” are used interchangeably and refer to a genetic or metabolic predisposition of a subject to develop a scoliosis (i.e. spinal deformity) and/or a more severe scoliosis at a future time.

As used herein the term “subject” is meant to refer to any mammal including human, mice, rat, dog, cat, pig, monkey, horse, etc. In a particular embodiment, it refers to a human. In an other particular embodiment, it refers to a horse and more specifically a racing horse.

As used herein the terms “blood sample” is whole blood and it is a cell sample in that it comprises peripheral blood mononuclear cells.

Without being so limited, cells where Pitx1 is known to be expressed include cells from muscles, bone and cartilages such as osteoblasts, chondrocytes and skeletal myoblasts.

The present invention also relates to methods for the determination of the level of expression of transcripts or translation product of a single gene such as pitxl. The present invention therefore encompasses any known method for such determination including real time PCR and competitive PCR, Northern blots, nuclease protection, plaque hybridization and slot blots.

The present invention also concerns isolated nucleic acid molecules including probes and primers to detect Pitx1. In specific embodiments, the isolated nucleic acid molecules have no more than 300, or no more than 200, or no more than 100, or no more than 90, or no more than 80, or no more than 70, or no more than 60, or no more than 50, or no more than 40 or no more than 30 nucleotides. In specific embodiments, the isolated nucleic acid molecules have at least 17, or at least 18, or at least 19, or at least 20, or at least 30, or at least 40 nucleotides. In other specific embodiments, the isolated nucleic acid molecules have at least 20 and no more than 300 nucleotides. In other specific embodiments, the isolated nucleic acid molecules have at least 20 and no more than 200 nucleotides. In other specific embodiments, the isolated nucleic acid molecules have at least 20 and no more than 100 nucleotides. In other specific embodiments, the isolated nucleic acid molecules have at least 20 and no more than 90 nucleotides. In other specific embodiments, the isolated nucleic acid molecules have at least 20 and no more than 80 nucleotides. In other specific embodiments, the isolated nucleic acid molecules have at least 20 and no more than 70 nucleotides. In other specific embodiments, the isolated nucleic acid molecules have at least 20 and no more than 60 nucleotides. In other specific embodiments, the isolated nucleic acid molecules have at least 20 and no more than 50 nucleotides. In other specific embodiments, the isolated nucleic acid molecules have at least 20 and no more than 40 nucleotides. In other specific embodiments, the isolated nucleic acid molecules have at least 17 and no more than 40 nucleotides. In other specific embodiments, the isolated nucleic acid molecules have at least 20 and no more than 30 nucleotides. In other specific embodiments, the isolated nucleic acid molecules have at least 17 and no more than 30 nucleotides. In other specific embodiments, the isolated nucleic acid molecules have at least 30 and no more than 300 nucleotides. In other specific embodiments, the isolated nucleic acid molecules have at least 30 and no more than 200 nucleotides. In other specific embodiments, the isolated nucleic acid molecules have at least 30 and no more than 100 nucleotides. In other specific embodiments, the isolated nucleic acid molecules have at least 30 and no more than 90 nucleotides. In other specific embodiments, the isolated nucleic acid molecules have at least 30 and no more than 80 nucleotides. In other specific embodiments, the isolated nucleic acid molecules have at least 30 and no more than 70 nucleotides. In other specific embodiments, the isolated nucleic acid molecules have at least 30 and no more than 60 nucleotides. In other specific embodiments, the isolated nucleic acid molecules have at least 30 and no more than 50 nucleotides. In other specific embodiments, the isolated nucleic acid molecules have at least 30 and no more than 40 nucleotides. It should be understood that in real-time PCR, primers also constitute probe without the traditional meaning of this term. Primers or probes appropriate to detect Pitx1 in the methods of the present invention can be designed with known methods using sequences distributed across the Pitx1 nucleotide sequence. (Buck et al. Design Strategies and Performance of Custom DNA Sequencing primers. Biotechniques 27:528-536 (September 1999)).

Although amino acid and nucleotide sequences for Pitx1 are included herein, the present invention is not so limited and encompasses the detection of any Pitx1 protein or nucleotides isolated from a subject. Without being so limited, the present invention encompasses the detection of the Pitx1 presented in Table 1.

TABLE 1
ACCESSION NUMBERS FOR PITX1 SEQUENCES

| Nucleotide | | Protein |
|---|---|---|
| Genomic | AC004764.1 | AAC17733.1 |
| Genomic | AC008406.7 | None |
| | (17530 . . . 24049, | |
| | complement) | |
| Genomic | AF009648.1 | AAB65251.1 |
| Genomic | AF009649.1 | AAB65251.1 |
| Genomic | AF009650.1 | AAB65251.1 |
| Genomic | CH471062.2 | EAW62226.1 |
| | | EAW62227.1 |
| Genomic | CS278249.1 | CAJ86537.1 |
| mRNA | AK290635.1 | BAF83324.1 |
| mRNA | AL578756.2 | None |
| mRNA | BC003685.1 | AAH03685.1 |
| mRNA | BC009412.1 | AAH09412.1 |
| mRNA | BX362641.2 | None |
| mRNA | CR601326.1 | None |
| mRNA | CR603120.1 | None |
| mRNA | CR610821.1 | None |
| mRNA | U70370.1 | AAC51126.1 |
| Synthetic | EU446647.1 | ABZ92176.1 |
| | | P78337.2 |
| mRNA | NM_002653 version | NP_002644 |
| | NM_002653.4 | version |
| | | NP_002644.4 |
| Chromosome 5, | NC_000005(134391323- | NC_000005.8 |
| reference assembly, | 134397863) | |
| complete sequence | | |
| Genomic | NC_000005.8 | |
| | Reference assembly | |
| | 134397863 . . . 134391323, | |
| | complement | |
| Genomic | NT_034772.5 | |
| | 36784977 . . . 36778437, | |
| | complement | |
| Genomic | AC_000048.1 | |
| | 130493751 . . . 130487209, | |
| | complement | |
| Genomic | NW_922784.1 | |
| | 8121767 . . . 8115225, | |
| | complement | |
| Genomic | AC_000137.1 | |
| | 91265592 . . . 91277386 | |
| Genomic | NW_001838952.2 | |
| | 4413502 . . . 4425296 | |
| Genomic | NW_001838952.2 | |
| | 4413502 . . . 4425296 | |

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally known (Miller, 1988. Ann. Reports Med. Chem. 23:295; Moran et al., 1987. Nucleic Acids Res., 14:5019.). Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

As used herein the terms “detectably labeled” refer to a marking of a probe or antibody in accordance with the presence invention that will allow the detection of the Pitx1 expression in methods and kits of the present invention. Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook, J., Fritsch, E. F. & Maniatis, T. (1989). Molecular Cloning: A Laboratory Manual, Cold Spring Harbor Laboratory Press, Cold Spring Harbor, N.Y.). Non-limiting examples of labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe or antibody.

As commonly known, radioactive nucleotides can be incorporated into probes of the invention by several methods. Non-limiting examples thereof include kinasing the 5′ ends of the probes using gamma 32P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

The present invention also relates to methods of selecting compounds. As used herein the term “compound” is meant to encompass natural, synthetic or semi-synthetic compounds, including without being so limited chemicals, macromolecules, cell or tissue extracts (from plants or animals), nucleic acid molecules, peptides, antibodies and proteins.

The present invention also relates to arrays. As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

As used herein “array of nucleic acid molecules” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Any known nucleic acid arrays can be used in accordance with the present invention. For instance, such arrays include those based on short or longer oligonucleotide probes or primers as well as cDNAs or polymerase chain reaction (PCR) products (Lyons P., 2003. Advances in spotted microarray resources for expression profiling. Briefings in Functional Genomics and Proteomics 2, 21-30). Other methods include serial analysis of gene expression (SAGE), differential display, (Ding G. and Cantor C. R., 2004. Quantitative analysis of nucleic acids—the last few years of progress. J Biochem Biol 37, 1-10) as well as subtractive hybridization methods (Scheel J., Von Brevern M. C., Horlein A., Fisher A., Schneider A., Bach A. 2002. Yellow pages to the transcriptome. Pharmacogenomics 3, 791-807), differential screening (DS), RNA arbitrarily primer (RAP)-PCR, restriction endonucleolytic analysis of differentially expressed sequences (READS), amplified restriction fragment-length polymorphisms (AFLP).

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (%GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see Sambrook, J., Fritsch, E. F. & Maniatis, T. (1989). Molecular Cloning: A Laboratory Manual, Cold Spring Harbor Laboratory Press, Cold Spring Harbor, N.Y. for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Washing with a solution containing tetramethylammonium chloride (TeMAC) could allow the detection of a single mismatch using oligonucleotide hybridyzation since such mismatch could generate a 10° C. difference in the annealing temperature. The formulation to determine the washing temperature is Tm (° C.)=]−682 (L⁻¹)+97 where L represents the length of the oligonucleotide that will be used for the hybridization.

The present invention relates to a kit for stratify AIS and/or predicting whether a subject is at risk of developing AIS comprising an isolated nucleic acid, a protein or a ligand such as an antibody in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the subject sample (DNA genomic nucleic acid, cell sample or blood samples), a container which contains in some kits of the present invention, the probes used in the methods of the present invention, containers which contain enzymes, containers which contain wash reagents, and containers which contain the reagents used to detect the extension products. Kits of the present invention may also contain instructions to use these probes and or antibodies to stratify AIS or predict whether a subject is at risk of developing AIS.

As used herein, the term “purified” in the expression “purified antibody” is simply meant to distinguish man-made antibody from an antibody that may naturally be produced by an animal against its own antigens. Hence, raw serum and hybridoma culture medium containing anti-Pitx1 antibody are “purified antibodies” within the meaning of the present invention.

As used herein, the term “ligand” broadly refers to natural, synthetic or semi-synthetic molecules. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The ligand appropriate for the present invention can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “ligand”. For example, peptidomimetics, well known in the pharmaceutical industry and generally referred to as peptide analogs can be generated by modeling as mentioned above.

### Antibodies

Both monoclonal and polyclonal antibodies directed to Pitx1 are included within the scope of this invention as they can be produced by well established procedures known to those of skill in the art. Additionally, any secondary antibodies, either monoclonal or polyclonal, directed to the first antibodies would also be included within the scope of this invention.

As used herein, the term “anti-Pitx1 antibody” or “immunologically specific anti-Pitx1 antibody” refers to an antibody that specifically binds to (interacts with) a Pitx1 protein and displays no substantial binding to other naturally occurring proteins other than the ones sharing the same antigenic determinants as the Pitx1 protein. The term antibody or immunoglobulin is used in the broadest sense, and covers monoclonal antibodies (including full length monoclonal antibodies), polyclonal antibodies, multispecific antibodies, and antibody fragments so long as they exhibit the desired biological activity. Antibody fragments comprise a portion of a full length antibody, generally an antigen binding or variable region thereof. Examples of antibody fragments include Fab, Fab′, F(ab′)₂, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (V_{H}, V_{H}-V_{H}), anticalins, PepBodies™, antibody-T-cell epitope fusions (Troybodies) or Peptibodies. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The term antibody encompasses herein polyclonal, monoclonal antibodies and antibody variants such as single-chain antibodies, humanized antibodies, chimeric antibodies and immunologically active fragments of antibodies (e.g. Fab and Fab′ fragments) which inhibit or neutralize their respective interaction domains in Hyphen and/or are specific thereto.

Polyclonal antibodies are preferably raised in animals by multiple subcutaneous (sc), intravenous (iv) or intraperitoneal (ip) injections of the relevant antigen with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N=C=NR, where R and R¹ are different alkyl groups.

Animals may be immunized against the antigen, immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293 (see also, e.g., Lindenbaum et al., 2004).

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 compares Pitx1 expression in osteoblasts from severely affected AIS patients and matched control subjects. Reverse transcription-polymerase chain reaction for pitx1 gene expression in human osteoblasts of control subjects (n=46) and patients with AIS (n=29). Pitx1 specific mRNA transcripts were detected in the control tissue. Loss of the pitxl gene expression was observed in all examined AIS samples and β-actin expression was used as internal control;

FIG. 2 shows the sequence of a 10 kb pitxl promoter region (SEQ ID NO: 1) and polymorphisms in that pitx1 promoter region between human subjects. The primers used to cover the different amplicons covering the 10 kb regions are provided in Table 4 below;

FIG. 3 shows the sequence of the Pitx1 mRNA (SEQ ID NO: 2) (NM_{—}002653); and

FIG. 4 shows the Pitx1 amino acid sequence (SEQ ID NO: 3) (NP_{—}002644).

### DESCRIPTION OF ILLUSTRATIVE EMBODIMENTS

The present invention demonstrated by RT-PCR analysis a loss of pitxl gene expression in osteoblasts derived from biopsies obtained intraoperatively of severely affected AIS patients (n=46) while osteoblasts derived from non-scoliotic patients (trauma cases) still expressed Pitx1 (n=29).

The present invention is illustrated in further details by the following non-limiting examples.

### Human Specimens

Informed consent was obtained from all study participants as approved by each individual and collective Institutional Review Board (Ste-Justine University Hospital, Montreal's Children Hospital and The Shriners Hospital for Children all located in Montreal). All individuals were screened through a series of steps including history and clinical data, assuring the idiopathic nature of the problem. This was followed by a review of spinal radiographs. A person was deemed to be affected by AIS if history and physical examination were consistent with the diagnosis of idiopathic scoliosis and a minimum of a ten degree curvature in the coronal plane with vertebral rotation was found on the radiograph. Participants will also be screened as to the potential familial distribution of the disorder on a case by case basis. In the event that the disorder is found to be familial, family members will undergo parallel screening for the presence or absence of the condition. Other patients (without scoliosis) visiting our trauma clinics were used as controls.

The clinical characteristics of the examined AIS and control subjects are shown in Table 2 and Table 3 respectively. Scoliotic patients with a diagnostic other than AIS were tested as control subjects.

TABLE 2
CLINICAL CHARACTERISTIC
OF EXAMINED AIS SUBJECTS

| | | | | Cobb | |
|---|---|---|---|---|---|
| Number | GeN/Aer | Age | Curve type | angle | Heredity |
| 1006 | f | 12, 65 | double major | 61-46 | No |
| 1007 | m | 18, 67 | right thoracolumbar | 61 | Yes |
| 1045 | f | 19, 48 | left thoracolumbar | 38 | No |
| 1066 | f | 17, 33 | right thoracic | 53 | Yes |
| 1137 | f | 20, 54 | double major | 65-42 | No |
| 1167 | m | 14, 58 | left thoracic | 49 | No |
| 1263 | f | 13, 32 | double major | 53 | No |
| 1266 | m | 15, 56 | double major | 52 | No |
| 1274 | f | 13, 27 | double major | 42 | No |
| 1276 | f | 15, 26 | left thoracic | 42 | No |
| 1277 | f | 12, 73 | double major | 57-48 | No |
| 1280 | f | 14, 4 | double major | 56-46 | No |
| 1294 | f | 16, 92 | left thoracic | N/A | Yes |
| 1306 | f | 13, 15 | double major | 77-48 | Yes |
| 1308 | f | 15, 3 | double major | 77-20 | No |
| 1310 | f | 15, 52 | double major | 55-42 | No |
| 1311 | f | 14, 6 | double major | 78 | Yes |
| 1315 | f | 14, 62 | right thoracic | 91 | No |
| 1317 | f | 13, 97 | right thoracic | 53 | Yes |
| 1318 | f | 13, 7 | left thoracolumbar | 49 | No |
| 1322 | f | 13, 11 | double major | 51 | No |
| 1325 | f | 16, 16 | left thoracic | 44 | Yes |
| 1329 | m | 14, 02 | right thoracolumbar | 61 | Yes |
| 1335 | f | 17, 6 | double major | 47-50 | No |
| 1337 | f | 14, 13 | double major | 57-48 | Yes |
| 1339 | f | 14, 28 | right thoracic | 31 | No |
| 1346 | f | 13, 54 | double major | 50-34 | Yes |
| 1347 | f | 18, 55 | double major | 56-45 | No |
| 1349 | f | 11, 69 | left thoracolumbar | 74 | No |
| 1352 | f | 7, 8 | right thoracic | 51 | No |
| 1360 | f | 9, 92 | double major | 53-46 | Yes |
| 1385 | f | 16, 06 | double major | 42-23 | No |
| 1390 | f | 15, 61 | left thoracolumbar | 53 | No |
| 1391 | f | 15, 01 | left lumbar | 54 | No |
| 1395 | f | 17, 79 | left thoracolumbar | 84 | Yes |
| 1402 | f | 15, 82 | right thoracic | 51 | No |
| 1406 | f | 14, 89 | double major | 62-60 | No |
| 1409 | f | 13, 62 | right thoracic | 40 | No |
| 1410 | f | 13, 73 | right thoracic | 56 | Yes |
| 1417 | f | 13, 24 | right thoracic | 59 | Yes |
| 1418 | f | 13, 08 | right thoracic | 41 | No |
| 1420 | f | 13, 42 | double major | 60-48 | Yes |
| 1422 | f | 12, 44 | double major | 60-50 | Yes |
| 1425 | f | 13, 42 | right thoracic | 68 | Yes |
| 1439 | f | N/A | right thoracic | 69 | Yes |
| 1442 | f | N/A | right thoracic | 60 | No |

N/A: not available

TABLE 3
CLINICAL CHARACTERISTICS OF EXAMINED CONTROL SUBJECTS

| Number | Gender | Age | Health status | Curve type | Cobb angle | Heredity |
|---|---|---|---|---|---|---|
| C100 | f | N/A | healthy | nd | nd | No |
| C101 | N/A | N/A | healthy | nd | nd | No |
| C102 | f | 18 | healthy | nd | nd | No |
| C103 | N/A | N/A | healthy | nd | nd | No |
| C104 | f | 14 | healthy | nd | nd | No |
| C105 | f | 11 | healthy | nd | nd | No |
| C106 | m | 12 | healthy | nd | nd | No |
| C107 | f | 13 | healthy | nd | nd | No |
| C108 | f | 12 | healthy | nd | nd | No |
| C109 | m | 14 | healthy | nd | nd | No |
| C110 | m | 15 | healthy | nd | nd | No |
| C111 | m | 14 | healthy | nd | nd | No |
| C112 | f | 11 | healthy | nd | nd | No |
| C113 | m | 14 | healthy | nd | nd | No |
| C114 | N/A | N/A | healthy | nd | nd | No |
| C115 | m | 17 | healthy | nd | nd | No |
| C116 | m | 12 | healthy | nd | nd | No |
| C117 | m | 16 | healthy | nd | nd | No |
| C118 | m | 12 | healthy | nd | nd | No |
| C119 | f | 15 | healthy | nd | nd | No |
| C120 | f | 15 | healthy | nd | nd | No |
| C121 | f | 8 | healthy | nd | nd | No |
| 1285 | f | 15, 87 | paralytic scoliosis | N/A | 72 | Yes |
| 1293 | m | 11, 79 | congenital scoliosis | left lumbar | 38 | No |
| 1341 | f | 11, 14 | congenital scoliosis | double major | 61-65 | No |
| 1375 | f | 13, 71 | congenital scoliosis | right thoracolumbar | 53 | Yes |
| 1431 | m | 19, 13 | neurological | double major | 90-90 | No |
| | | | scoliosis | | | |
| 1434 | f | 12, 43 | congenital scoliosis | double major | 79-77 | No |
| 1436 | f | 13, 93 | kyphoscoliosis | kyphosis | 120 | No |

N/A: not available and
nd: not detected

### Osteoblasts Cultures

Osteoblasts were obtained from bone specimens taken intraoperatively during spine surgeries of AIS patients and trauma surgeries in the case of control subjects. This cell type was chosen as cellular model for this study as described previously (Rodriguez M M, Ron D, Touhara K, Chen C H, Mochly-Rosen D. RACK1, a protein kinase C anchoring protein, coordinates the binding of activated protein kinase C and select pleckstrin homology domains in vitro. Biochemistry. 1999;38:13787-13794).

### Isolation of Human Osteoblasts

In all cases, osteoblasts were obtained intraoperatively from bone specimens originating from vertebras (varying from T3 to L4 according to the surgical procedure performed). Bony fragments were mechanically reduced to smaller pieces with a bone cutter in sterile conditions and incubated at 37° C. in 5% CO₂ in a 100 mm culture dish in presence of DMEM medium containing 10% fetal bovine serum (FBS) (certified FBS, Invitrogen, Burlington, ON, Canada) and 1% penicillin/streptomycin (Invitrogen). After a 30-day period, the osteoblasts derived from the bone pieces were separated at confluence from the remaining bone fragments by trypsinization.

### Total RNA Isolation and RT-PCR

Extraction of RNA from osteoblasts was done using the standard Trisol Reagent method. (Invitrogen).

The RNA obtained from the osteoblasts was used for cDNA synthesis performed with the Invitrogen Thermoscript™ RT-PCR system and the respective protocol in the following conditions: Enzyme used: Taq DNA polymerase from Invitrogen™. PCR conditions: 95° C. 5 minutes, Hot start (1 cycle). Following three reactions (32 cycles): 94° C., 45 Seconds Denaturation; 55° C. 45 Seconds; Primer annealing; 72° C. 1 minute Elongation; 72° C. 2 minutes Last elongation (1 cycle); 4° C. 20 minutes pause; Duration: 2 hours 42 minutes. The quality of the cDNA was tested by amplifying 233 bp fragment of human beta-actin using the sense primer 5′-GGAAATCGTGCGTGACAT-3′ (SEQ ID NO: 4) and antisense primer 5′-TCATGATGGAGTTGAATGTAGTT-3′ (SEQ ID NO: 5). For quantitative analysis, all amplifications were normalized against that of the housekeeping gene β-actin. PCR amplified product were separated on 1.5% agarose gel and visualized by ethidium bromide staining.

### Expression Analysis of Pitx1:

Coding region of Pitx1 501 by in length was amplified from the cDNA using the sense primer 5′-GACCCAGCCAAGAAGAAGAA-3′ (SEQ ID NO: 6) and the antisense primer 5′-GAGGTTGTTGATGTTGTTGAGG-3′ (SEQ ID NO: 7) under the following PCR conditions: Enzyme used: Taq DNA polymerase from Invitrogen™. PCR conditions: 95° C. 10 minutes hot start (1 cycle); Following three reactions (34 cycles): 94° C. 45 Seconds Denaturation; 69° C. 45 Seconds Primer annealing; 72° C. 1 minute Elongation; 72° C. 2 minutes Last elongation (1 cycle); 4° C. 20 minutes; 4° C. Pause; Duration: 2hours 34 minutes 11 seconds.

### Pitx1 Promoter Sequencing

10 kb region of the pitxl promoter was amplified and sequenced to screen for mutations. Enzyme used: Platinum® Taq DNA polymerase High fidelity from Invitrogen™ under the following conditions: 95° C. 5 minutes hot start (1 cycle). Following three reactions 35 cycles: 94° C. 45 Seconds Denaturation; 61.8° C. 45 Seconds Primer annealing; 72° C. 1 minute Elongation; 72° C. 5 minutes Last elongation (1 cycle); 4° C. 20 minutes; and 4° C. Pause.

One hundred (100) ng of genomic DNA was mixed in a final volume of 25 μl containing 200 micromolar dNTPs, 1,5 mM MgCl₂, 10 pM of each primer (see Table 4 below for list of primers used), and 1U Pfx DNA-polymerase (Invitrogen) or an other DNA polymerase.

PCR conditions: Regions PP1, PP2, PP3, PP6, PP7 were amplified using Platinum® pfx DNA polymerase from Invitrogen™ under the following PCR conditions: 95° C. 5 minutes hot start (1 cycle); Following three reactions (35 cycles): 94° C. 30 Seconds Denaturation; 60° C. 30 Seconds; Primer annealing; 68° C. 1 min 20 Sec Elongation; 68° C. 2 minutes; Last elongation (1 cycle); 4° C. 20 minutes; 4° C. Pause. Duration: 2 hours 35 minutes 26 seconds

Regions PP4, PPS, PPB, PP9 and PP10 were amplified using Platinum® Taq DNA polymerase High fidelity from Invitrogen™ under the following conditions. 95° C. 2 minutes hot start (1 cycle); Following three reactions (35 cycles); 94° C. 45 Seconds Denaturation; 60° C. 45 Seconds Primer annealing; 72° C. 1 min 20 Sec Elongation; 72° C. 5 minutes Last elongation (1 cycle); 4° C. 20 minutes; 4° C. Pause; Duration: 2 hours 53 minutes 4 seconds.

TABLE 4
PITX PROMOTER PRIMERS

| | | |
|---|---|---|
| PP1 (962 bp) | forward | 5′-CTGTTTGCTCAAGACGCTGA-3′; |
| | primer | (SEQ ID NO: 8) |
| | reverse | 5′-CTCGGCCTCACAAAAGAAAC-3′ |
| | primer | (SEQ ID NO: 9) |
| PP2 (966 bp) | forward | 5′-TGTCTGCATTCAGGCTGTTC-3′; |
| | primer | (SEQ ID NO: 10) |
| | reverse | 5′-GATTCCCTCCTCGAGTCCTT-3′ |
| | primer | (SEQ ID NO: 11) |
| PP3 (1039 bp) | forward | 5′-CAAGTGAGCTGGATGCTGAA-3′; |
| | primer | (SEQ ID NO: 12) |
| | reverse | 5′-AGGGAGTGTCCCTTCACAGA-3′ |
| | primer | (SEQ ID NO: 13) |
| PP4 (1085 bp) | forward | 5′-GCTCAGCCATTCTCAGGAAC-3′; |
| | primer | (SEQ ID NO: 14) |
| | reverse | 5′-GCCATTGTCCCAGTCAAGAT-3′ |
| | primer | (SEQ ID NO: 15) |
| PP5 (1011 bp) | forward | 5′-TCGCGTCAAGAGGGTATTTT-3′; |
| | primer | (SEQ ID NO: 16) |
| | reverse | 5′-TAGGACCCATGGCTCTACCC-3′ |
| | primer | (SEQ ID NO: 17) |
| PP6 (1098 bp) | forward | 5′-CACGAGTCAGGTGGGAAACT-3′; |
| | primer | (SEQ ID NO: 18) |
| | reverse | 5′-GACGTCTGCTGCTTTTCTGC-3′ |
| | primer | (SEQ ID NO: 19) |
| PP7 (963 bp) | forward | 5′-AGGCACGGACTAGCAGGAC-3′; |
| | primer | (SEQ ID NO: 20) |
| | reverse | 5′-ATGCGGACGAAGCCAGAG-3′ |
| | primer | (SEQ ID NO: 21) |
| PP8 (986 bp) | forward | 5′-TTAGCATTCAGCCCCTCTGT-3′; |
| | primer | (SEQ ID NO: 22) |
| | reverse | 5′-TTCATGAGATGCAGTCAGCAG-3′ |
| | primer | (SEQ ID NO: 23) |
| PP9 (951 bp) | forward | 5′-ACAACTGGTAGGGGCAACAG-3′; |
| | primer | (SEQ ID NO: 24 |
| | reverse | 5′-TGTGTGGCTTTGGCAAATAA-3′ |
| | primer | (SEQ ID NO: 25) |
| PP10 (990 bp) | forward | 5′-GCACTGTGCTCCAACTGTGT-3′; |
| | primer | (SEQ ID NO: 26) |
| | reverse | 5′-GGGGGAGTGTTCTTTTCCTT-3′ |
| | primer | (SEQ ID NO: 27) |

### EXAMPLE 1

### Comparison of Pitx1 Expression in Osteoblasts of AIS Subjects with that in Osteoblast of Matched Controls

To determine whether pitxl plays a role in the genetic control of AIS development and/or progression, an expression analysis of pitxl gene using RNA prepared from osteoblasts cultures derived from biopsies obtained intraoperatively of severely affected AIS patients (n=46) and from non-scoliotic patients (trauma cases) control subjects (n=29) was performed.

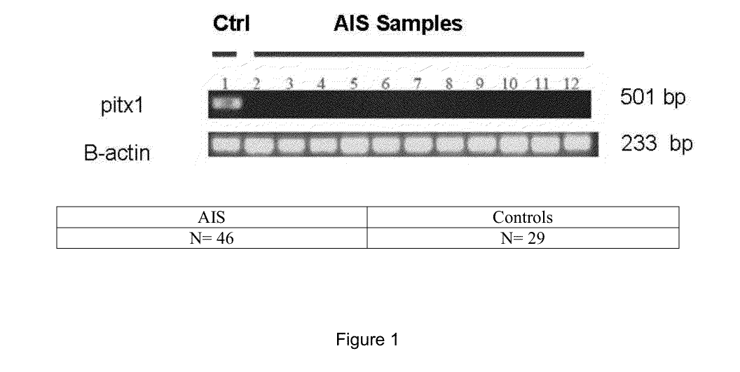

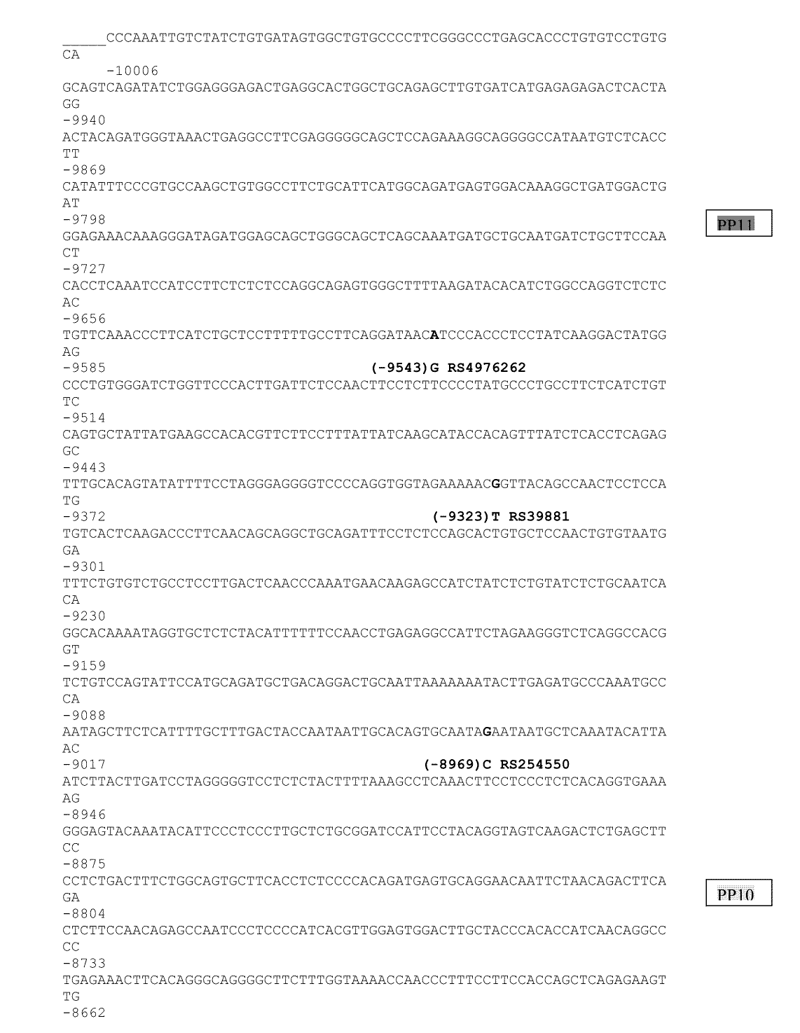

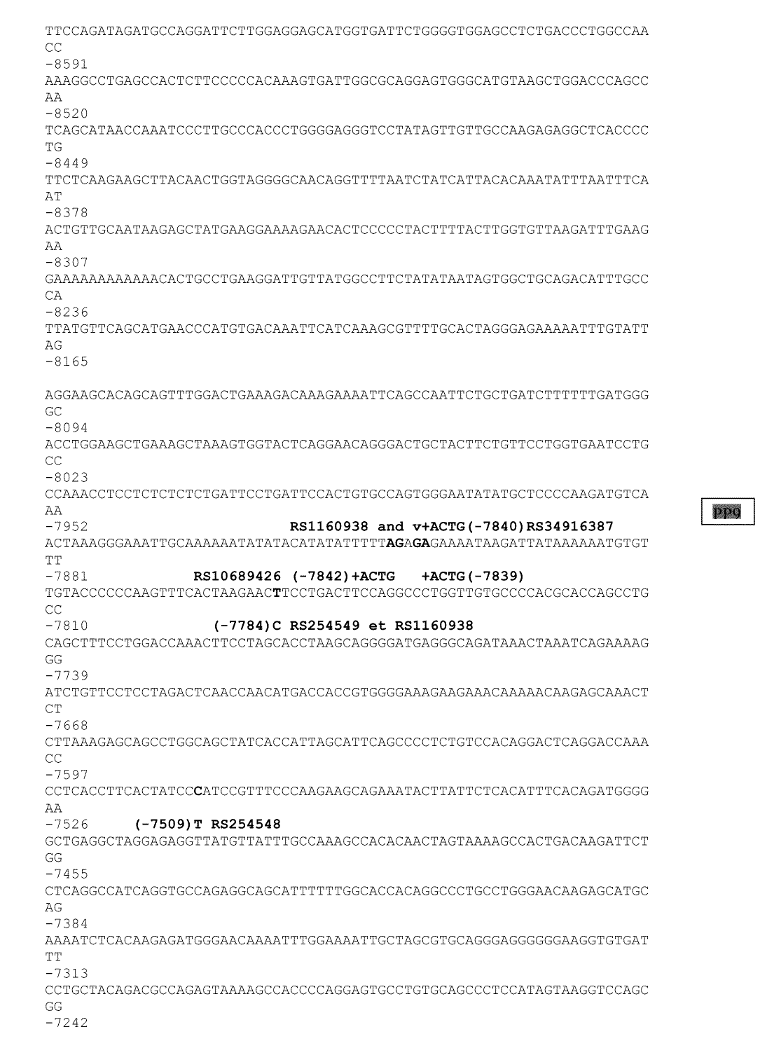

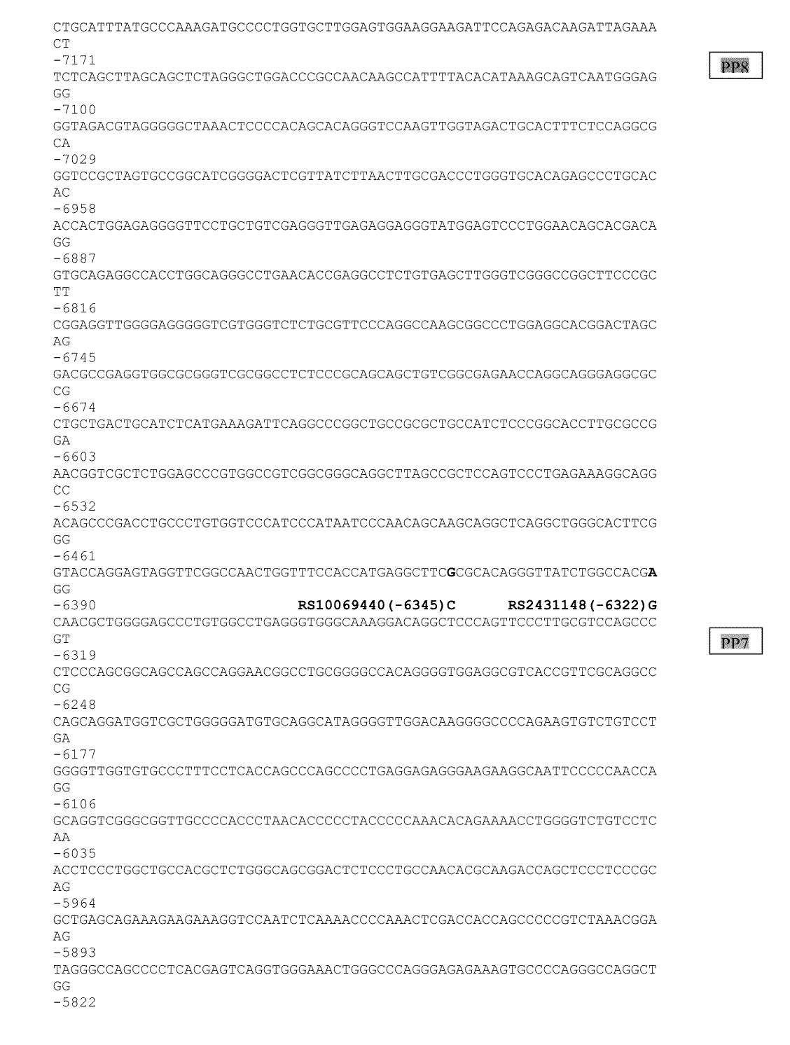

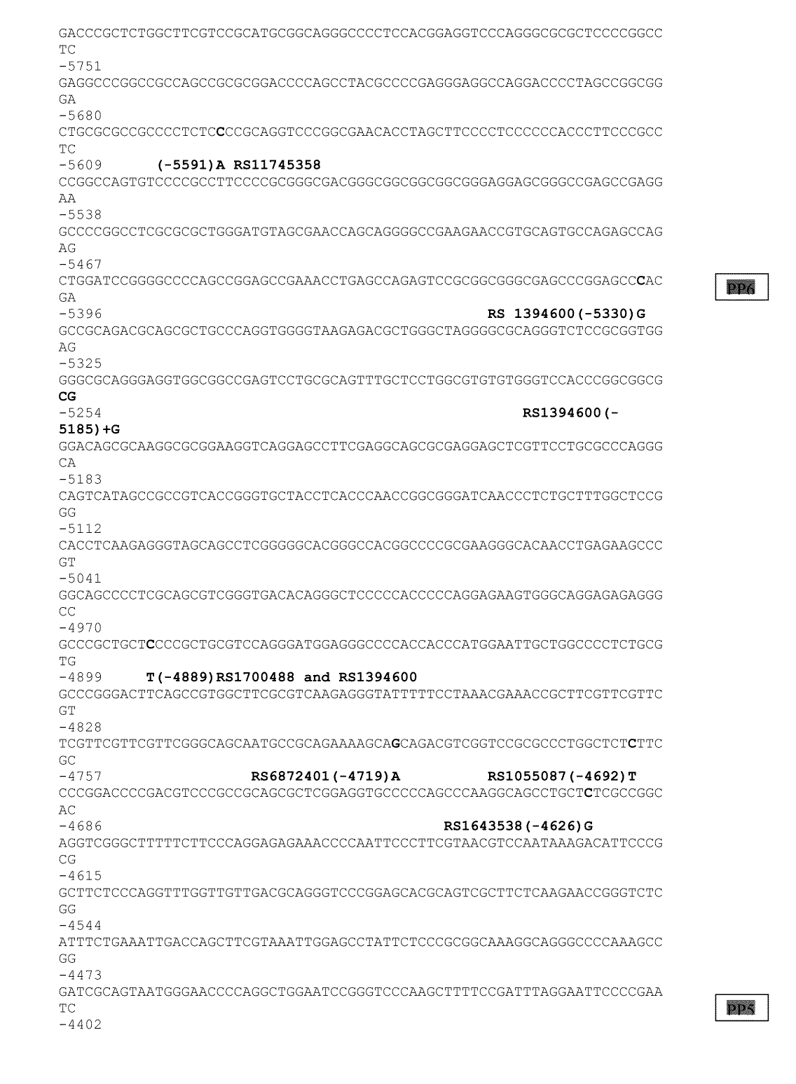

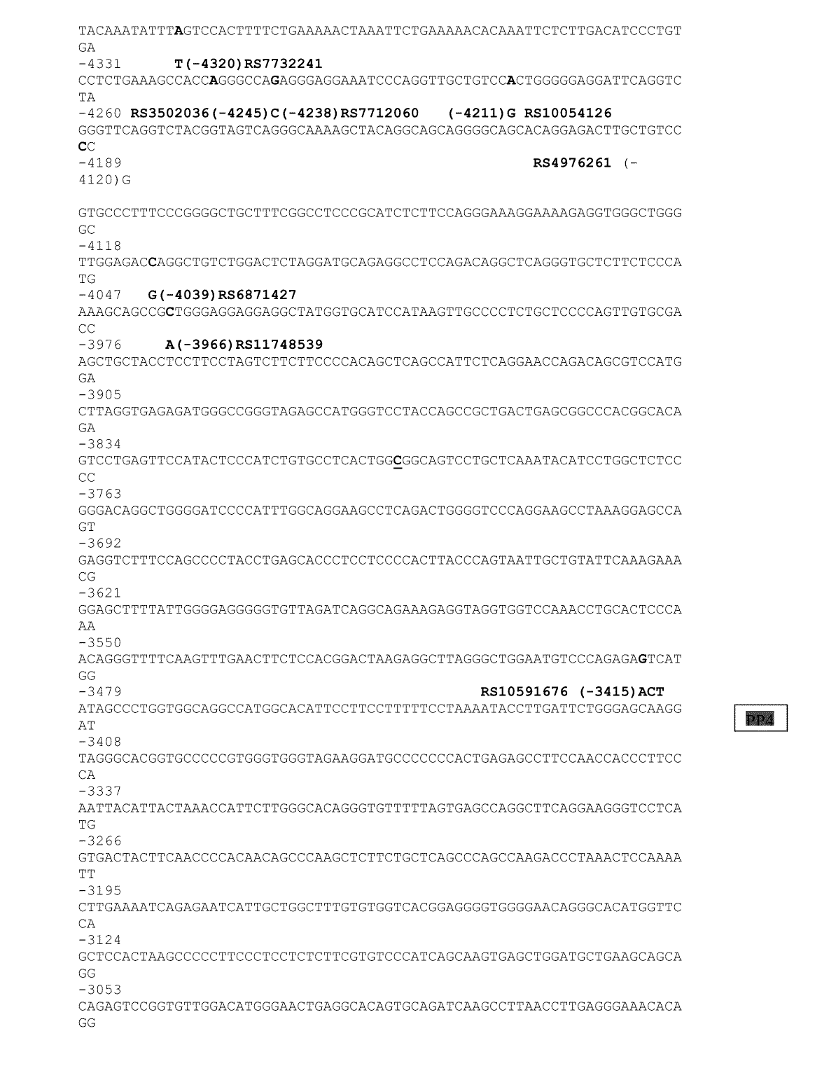

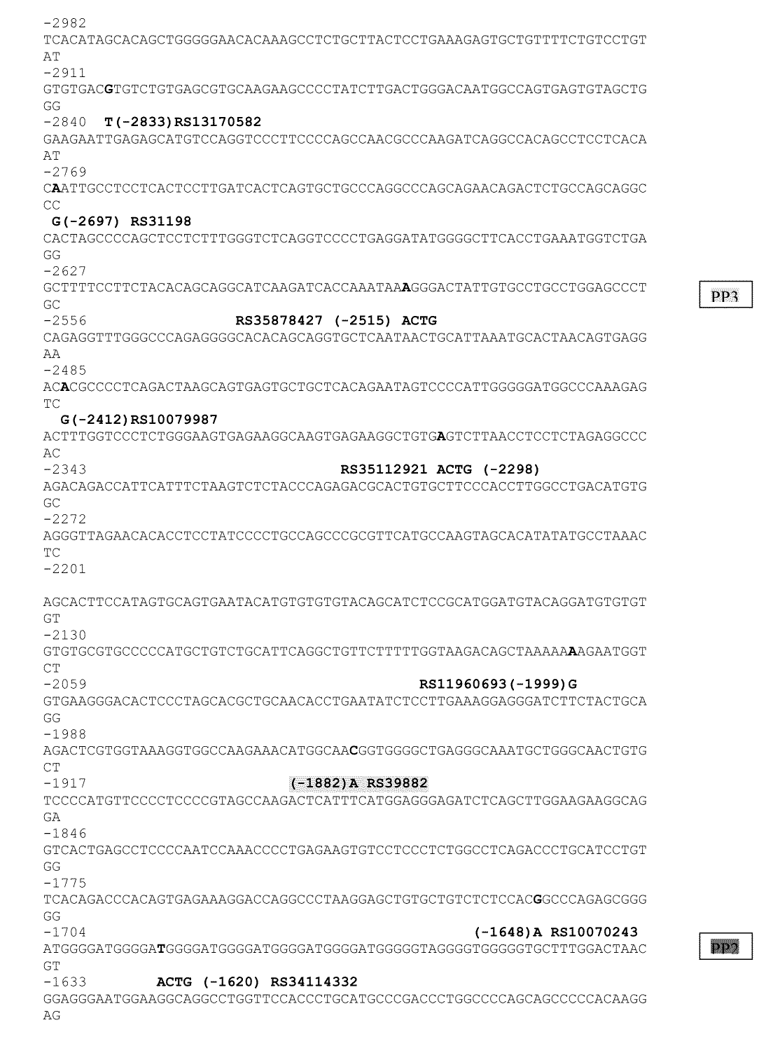

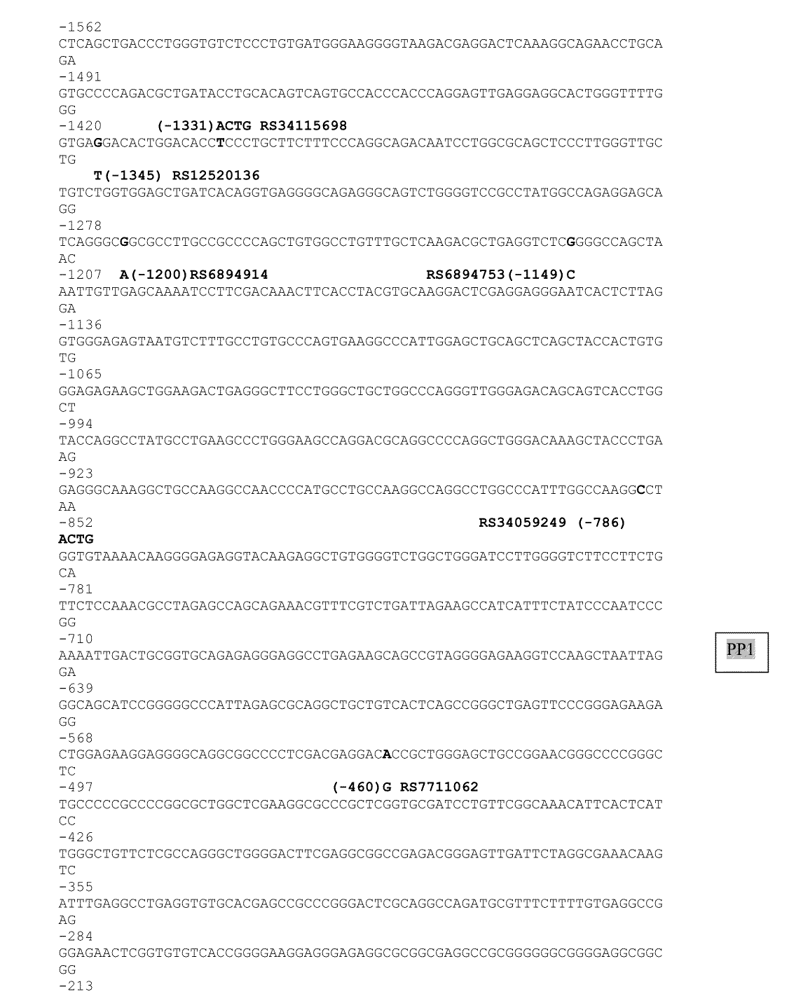

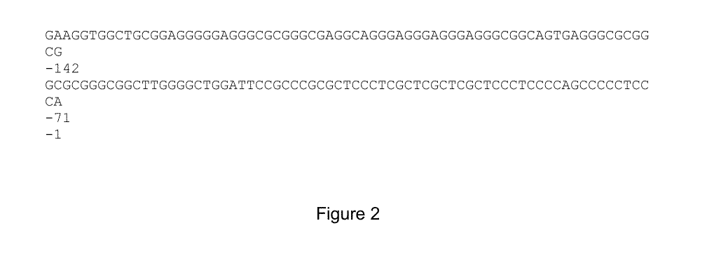

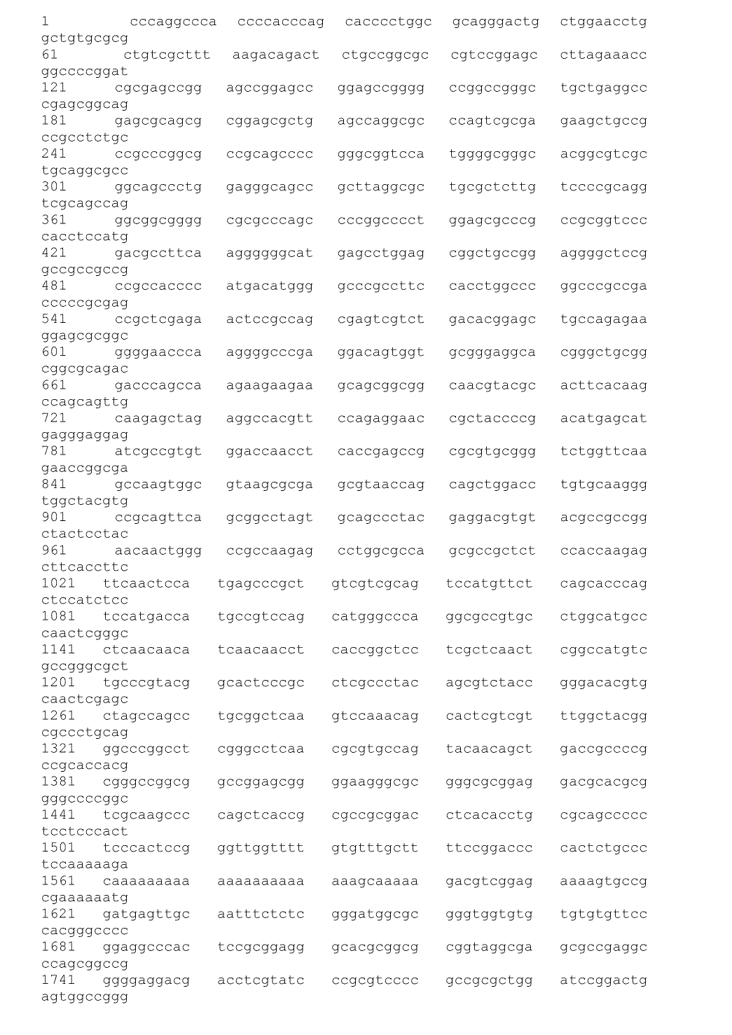

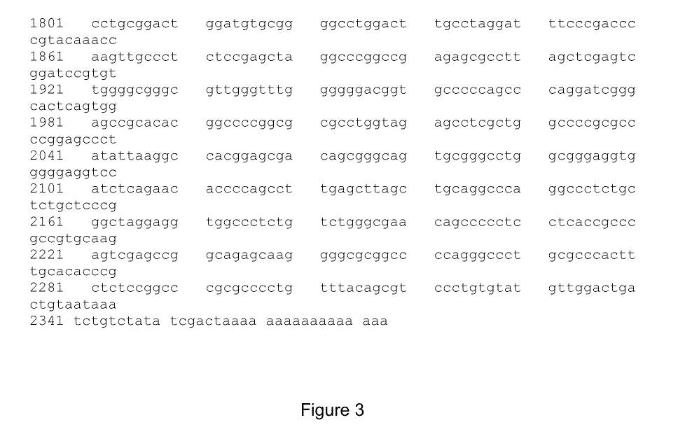

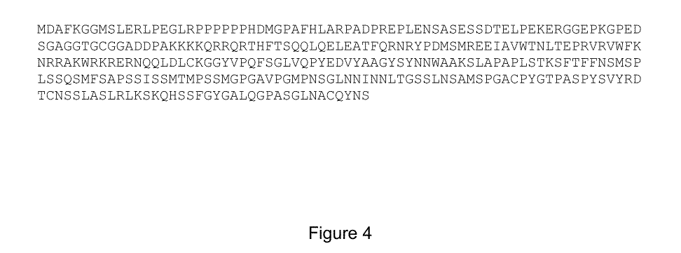

As may be seen in FIG. 1, all osteoblasts derived from the AIS patients showed a loss of pitxl mRNA expression, while control subjects still expressed the mRNA.

### EXAMPLE 2

### Determination of Pitx1 Expression in a Subject Sample

Tissue such as muscle (using for instance a needle in the paraspinal region), bone, cartilage, peripheral blood mononuclear cells (PBMCs such as T and B lymphocytes as well as macrophages) or any cells derived from tissues where Pitx1 is expressed is isolated from the patient. Extraction of RNA from these tissues is done using any standard RNA extraction method such as the standard Trisol Reagent method. Coding region of Pitx1 501 bp in length is amplified from the cDNA using for instance RT-PCR or real-time PCR. The sense primer 5′-GACCCAGCCAAGAAGAAGAA-3′ (SEQ ID NO: 6) and the antisense primer 5′-GAGGTTGTTGATGTTGTTGAGG-3′ (SEQ ID NO: 7) under the following PCR conditions: Enzyme used: Taq DNA polymerase from Invitrogen™. PCR conditions: 95° C. 5minutes Hot start (1 cycle). Following three reactions (32 cycles): 94° C. 45 Seconds Denaturation; 55° C. 45 Seconds Primer annealing; 72° C. 1 minute Elongation; 72° C. 2 minutes Last elongation (1 cycle); and 4° C. 20 minutes pause. Duration: 2 hours 42 minutes.

Although the present invention has been described herein above by way of specific embodiments thereof, it can be modified, without departing from the spirit and nature of the subject invention as defined in the appended claims.

1. A method for predicting a risk for developing adolescent idiopathic scoliosis (AIS) in a human subject comprising
(a) providing a cell sample isolated from the subject;
(b) detecting Paired-like homeodomain transcription factor 1 (Pitx1) expression in the cell sample; and
(c) determining that said subject has an increased risk for developing AIS when a loss of Pitx1 expression relative to Pitx1 expression in a cell sample from a non-scoliotic subject is detected in said cell sample,
wherein the cell sample is an osteoblasts sample, a chondrocytes sample, a skeletal myoblasts sample or a blood sample comprising peripheral blood mononuclear cells (PBMCs).

2. The method of claim 1, wherein a loss of Pitx1 expression is indicative that the subject is at risk for developing a Cobb's angle of 45° and above.

3. The method of claim 1, wherein said subject is pre-diagnosed as being a likely candidate for developing AIS.

4. The method of claim 1, wherein the detecting step is performed with an antibody that binds specifically to Pitx1.

5. The method of claim 1, wherein the cell sample is an osteoblasts sample.

6. The method of claim 1, further comprising a step of selecting a preventive action or a treatment in light of the results of the detecting step.

7. The method of claim 1, wherein said risk of developing AIS is a risk of requiring corrective surgery.

8. The method of claim 1, wherein the detecting step is performed with an isolated nucleic acid molecule specific to a Pitx1 transcription product.

9. The method of claim 8, wherein the isolated nucleic acid molecule is detectably labeled.

10. A method of selecting a compound potentially useful in the treatment of adolescent idiopathic scoliosis, said method comprising:
(a) contacting a test compound with at least one cell known to express Paired-like homeodomain transcription factor 1 (Pitx1); and
(b) determining Pitx1 expression level,
wherein the test compound is selected if Pitx1 expression level is increased in the presence of the test compound as compared to that in the absence thereof.

11. The method of claim 10, wherein said cell is an osteoblast.

12. The method of claim 10, wherein said cell is from a subject having adolescent idiopathic scoliosis (AIS).
